SEQUENCE LISTING (39 sequences)

SEQ ID NO: 1 - protein, 11 aa, Artificial Sequence
  MHC class I peptide
AMLKCVAFCDE

SEQ ID NO: 2 - protein, 11 aa, Artificial Sequence
  MHC class I peptide
ANGCSTIAQAV

SEQ ID NO: 3 - protein, 9 aa, Artificial Sequence
  MHC class I peptide
AQGVFGVNM

SEQ ID NO: 4 - protein, 12 aa, Artificial Sequence
  MHC class I peptide
ARLEPCNGTDID

SEQ ID NO: 5 - protein, 10 aa, Artificial Sequence
  MHC class I peptide
AVTTGDVKIM

SEQ ID NO: 6 - protein, 15 aa, Artificial Sequence
  MHC class I peptide
DIVVVDEVSMCTNYD

SEQ ID NO: 7 - protein, 11 aa, Artificial Sequence
  MHC class I peptide
DSLCAKAVTAY

SEQ ID NO: 8 - protein, 13 aa, Artificial Sequence
  MHC class I peptide
EDFLNMDIGVFIQ

SEQ ID NO: 9 - protein, 15 aa, Artificial Sequence
  MHC class I peptide
EVNADIVVVDEVSMC

SEQ ID NO: 10 - protein, 10 aa, Artificial Sequence
  MHC class I peptide
FVGADGELPV

SEQ ID NO: 11 - protein, 12 aa, Artificial Sequence
  MHC class I peptide
FVKSICNSAVAV

SEQ ID NO: 12 - protein, 15 aa, Artificial Sequence
  MHC class I peptide
FYCTNNTLVSGDAHI

SEQ ID NO: 13 - protein, 12 aa, Artificial Sequence
  MHC class I peptide
GAKVVNANVLTK

SEQ ID NO: 14 - protein, 9 aa, Artificial Sequence
  MHC class I peptide
GYIADISAF

SEQ ID NO: 15 - protein, 15 aa, Artificial Sequence
  MHC class I peptide
IACSKSARLKRFPVN

SEQ ID NO: 16 - protein, 11 aa, Artificial Sequence
  MHC class I peptide
IADFLAGSSDV

SEQ ID NO: 17 - protein, 9 aa, Artificial Sequence
  MHC class I peptide
IFAQTSDTA

SEQ ID NO: 18 - protein, 10 aa, Artificial Sequence
  MHC class I peptide
IVQMIADFLA

SEQ ID NO: 19 - protein, 14 aa, Artificial Sequence
  MHC class I peptide
KFLNAFDVFVTAIQ

SEQ ID NO: 20 - protein, 10 aa, Artificial Sequence
  MHC class I peptide
LIAGKLLPPV

SEQ ID NO: 21 - protein, 13 aa, Artificial Sequence
  MHC class I peptide
LNCALGAFAIFCC

SEQ ID NO: 22 - protein, 8 aa, Artificial Sequence
  MHC class I peptide
MHGVTLKI

SEQ ID NO: 23 - protein, 14 aa, Artificial Sequence
  MHC class I peptide
MKVKATKGEGDGGI

SEQ ID NO: 24 - protein, 9 aa, Artificial Sequence
  MHC class I peptide
NAMLKCVAF

SEQ ID NO: 25 - protein, 11 aa, Artificial Sequence
  MHC class I peptide
NEADYRCACYA

SEQ ID NO: 26 - protein, 11 aa, Artificial Sequence
  MHC class I peptide
PNLNLGILQVT

SEQ ID NO: 27 - protein, 13 aa, Artificial Sequence
  MHC class I peptide
PSLVMPPSPSPLV

SEQ ID NO: 28 - protein, 14 aa, Artificial Sequence
  MHC class I peptide
QAAAAMYKEARAVN

SEQ ID NO: 29 - protein, 15 aa, Artificial Sequence
  MHC class I peptide
QTSQALQTVATALNK

SEQ ID NO: 30 - protein, 14 aa, Artificial Sequence
  MHC class I peptide
SEISANGCSTIAQA

SEQ ID NO: 31 - protein, 13 aa, Artificial Sequence
  MHC class I peptide
SNFNTLFATTIPN

SEQ ID NO: 32 - protein, 11 aa, Artificial Sequence
  MHC class I peptide
TIQGPPGSGKS

SEQ ID NO: 33 - protein, 12 aa, Artificial Sequence
  MHC class I peptide
TNVPLQVGFSNG

SEQ ID NO: 34 - protein, 12 aa, Artificial Sequence
  MHC class I peptide
TNVPLQVGFSNG

SEQ ID NO: 35 - protein, 13 aa, Artificial Sequence
  MHC class I peptide
VLFSATAVKTGGK

SEQ ID NO: 36 - protein, 11 aa, Artificial Sequence
  MHC class I peptide
VLNNGFGGKQI

SEQ ID NO: 37 - protein, 12 aa, Artificial Sequence
  MHC class I peptide
VTSGLGTVDADY

SEQ ID NO: 38 - protein, 5 aa, Artificial Sequence
  Peptide portion of linker
FLAAY

SEQ ID NO: 39 - protein, 4 aa, Artificial Sequence
  Peptide portion of linker
LAAY

(19) United States
(12) Patent Application Publication
(10) Pub. No.: US 2024/0269264 A1
(43) Pub. Date: Aug. 15, 2024

(54) HUMAN CORONAVIRUS 229E DERIVED PEPTIDES

(71) Applicant: EMERGEX VACCINES HOLDING LIMITED, Abingdon, Oxfordshire (GB)

(72) Inventors: Thomas Rademacher, Abingdon, Oxfordshire (GB); Xiaofang Huang, Jenkintown, PA (US)

(21) Appl. No.: 18/564,886

(22) PCT Filed: Jun. 1, 2022
(86) PCT No.: PCT/EP2022/064962

§ 371 Date: Nov. 28, 2023

Related U.S. Application Data
(60) Provisional application No. 63/196,121, filed on Jun. 2, 2021.

(51) Int. Cl. A61K 39/215; A61K 39/00; C07K 14/005; C12N 7/00

(52) U.S. Cl. CPC A61K 39/215; C07K 14/005; C12N 7/00; A61K 2039/53; A61K 2039/55555; C12N 2770/20022; C12N 2770/20034

(57) ABSTRACT

The invention relates to common cold coronavirus peptides derived from HcoV-229E, and related polynucleotides, complexes pharmaceutical compositions, methods of treatment, and medical uses.

This application claims priority from U.S. 63/196,121 filed 2 Jun. 2021, the contents and elements of which are herein incorporated by reference for all purposes.

### FIELD OF THE INVENTION

The invention relates to common cold coronavirus peptides, and related polynucleotides, complexes pharmaceutical compositions, methods of treatment, and medical uses.

### BACKGROUND

Coronaviruses are a group of related viruses that cause diseases in mammals and birds. Symptoms of coronavirus infections vary between species. For instance, coronavirus infection in chickens causes upper respiratory tract disease, whereas coronavirus infections in cows and pigs tend to cause diarrhoea.

In humans, coronaviruses cause respiratory tract infections. The disease caused by infection with some coronaviruses can be mild, such as the common cold. Other coronaviruses cause more serious and potentially fatal disease, such as SARS, MERS, and COVID-19. No vaccine for any common cold coronavirus is yet commercially available. The provision of immunogenic peptides that could be used in such vaccines and/or for research purposes is highly desirable.

### SUMMARY OF THE INVENTION

The present invention relates to immunogenic peptides derived from a common cold coronavirus (CCC). The peptides may be used to prevent or treat common cold coronavirus infections in humans. The peptides may also find utility in common cold coronavirus research.

In more detail, the present inventors have identified number of peptides that are presented by MHC molecules on cells infected with the common cold coronavirus 229E. The peptides may be conserved between 229E and other common cold coronaviruses such as NL63, OC43, and HKU1. Inclusion of one or more such peptides in a pharmaceutical composition may confer protective capability against one or more common cold coronaviruses, and/or the ability to treat an existing common cold coronavirus infection. Furthermore, a complex comprising one or more such peptides bound to a MHC molecule may be used in the diagnosis of, or research into, common cold coronavirus infection.

Accordingly, the present invention provides a peptide comprising a sequence selected from SEQ ID NOs: 1 to 37 or a variant of any thereof.

The present invention also provides:
a polynucleotide encoding the peptide of the invention;
a pharmaceutical composition comprising a peptide of the invention;
a method of preventing or treating a common cold coronavirus infection in an individual, comprising administering the pharmaceutical composition of the invention to the individual
the pharmaceutical composition of the invention for use in a method of preventing or treating a common cold coronavirus infection in an individual, the method comprising administering the pharmaceutical composition to the individual;
the use of the pharmaceutical composition of the invention in the preparation of a medicament for use in a method of preventing or treating a common cold coronavirus (CCC) infection in an individual; and
a complex comprising a peptide of the invention bound to a MHC molecule.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1: Summary of HcoV229E derived peptide in control and infection groups.

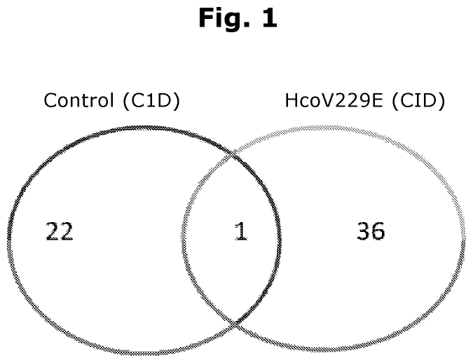

FIG. 2: Summary of self-derived peptides in control and infection groups.

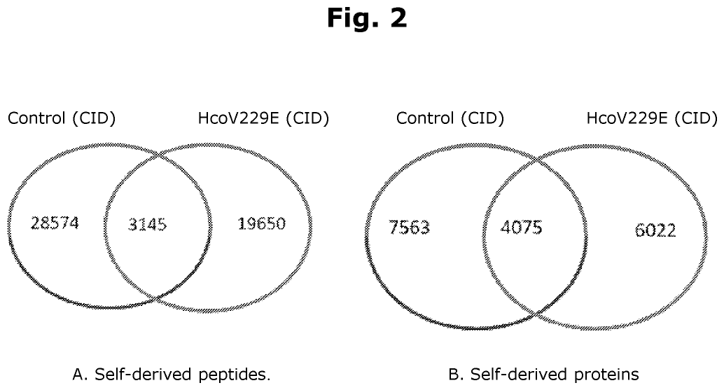

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a polynucleotide” includes “polynucleotides”, reference to “an amino acid” includes two or more such amino acids, and the like.

In general, the term “comprising” is intended to mean including but not limited to. For example, the phrase “peptide comprising a sequence selected from SEQ ID NOs: 1 to 37” should be interpreted to mean that the peptide contains a sequence selected from SEQ ID NOs: 1 to 37, but that the peptide may also contain additional amino acids.

In some aspects of the disclosure, the word “comprising” is replaced with the phrase “consisting of”. The term “consisting of” is intended to be limiting. For example, the phrase “a peptide consisting of a sequence selected from SEQ ID NOs: 1 to 37” should be understood to mean that the peptide contains a sequence selected from SEQ ID NOs: 1 to 37 and no additional amino acids.

For the purpose of this disclosure, in order to determine the percent identity of two sequences (such as two polynucleotide or two amino acid sequences), the sequences are aligned for optimal comparison purposes (e.g., gaps can be introduced in a first sequence for optimal alignment with a second sequence). The nucleotide/amino acid residues at nucleotide/amino acid positions are then compared. When a position in the first sequence is occupied by the same nucleotide or amino acid residue as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm.

### Peptides

The invention provides a peptide comprising a sequence selected from SEQ ID NOs: 1 to 37 or a variant of any thereof. Variants are defined in detail below. SEQ ID NOs: 1 to 37 are set out in Table 1.

TABLE 1

| SEQ | | Protein source | Viral |
|---|---|---|---|
| ID NO: | Sequence | NCBI ref. seq. | origin |
| 1 | AMLKCVAFCDE | NCBI ref. seq. NP_073549.1 | 229E |
| 2 | ANGCSTIAQAV | NCBI ref. seq. NP_073549.1 | 229E |
| 3 | AQGVFGVNM | NCBI ref. seq. NP_073549.1 | 229E |
| 4 | ARLEPCNGTDID | NCBI ref. seq. NP_073549.1 | 229E |
| 5 | AVTTGDVKIM | NCBI ref. seq. NP_073549.1 | 229E |
| 6 | DIVVVDEVSMCTNYD | NCBI ref. seq. NP_073549.1 | 229E |
| 7 | DSLCAKAVTAY | NCBI ref. seq. NP_073549.1 | 229E |
| 8 | EDFLNMDIGVFIQ | NCBI ref. seq. NP_073549.1 | 229E |
| 9 | EVNADIVVVDEVSMC | NCBI ref. seq. NP_073549.1 | 229E |
| 10 | FVGADGELPV | NCBI ref. seq. NP_073549.1 | 229E |
| 11 | FVKSICNSAVAV | NCBI ref. seq. NP_073549.1 | 229E |
| 12 | FYCTNNTLVSGDAHI | NCBI ref. seq. NP_073549.1 | 229E |
| 13 | GAKVVNANVLTK | NCBI ref. seq. NP_073549.1 | 229E |
| 14 | GYIADISAF | NCBI ref. seq. NP_073549.1 | 229E |
| 15 | IACSKSARLKRFPVN | NCBI ref. seq. NP_073549.1 | 229E |
| 16 | IADFLAGSSDV | NCBI ref. seq. NP_073549.1 | 229E |
| 17 | IFAQTSDTA | NCBI ref. seq. NP_073549.1 | 229E |
| 18 | IVQMIADFLA | NCBI ref. seq. NP_073549.1 | 229E |
| 19 | KFLNAFDVFVTAIQ | NCBI ref. seq. NP_073549.1 | 229E |
| 20 | LIAGKLLPPV | NC_002645_Human_coronavirus | 229E |
| | | _229E_Translation_RF-1_24 | |
| 21 | LNCALGAFAIFCC | NCBI ref. seq. NP_073549.1 | 229E |
| 22 | MHGVTLKI | NCBI ref. seq. NP_073549.1 | 229E |
| 23 | MKVKATKGEGDGGI | NCBI ref. seq. NP_073549.1 | 229E |
| 24 | NAMLKCVAF | NCBI ref. seq. NP_073549.1 | 229E |
| 25 | NEADYRCACYA | NCBI ref. seq. NP_073549.1 | 229E |
| 26 | PNLNLGILQVT | NC_002645_Human_coronavirus | 229E |
| | | _229E_Translation_RF+1_20 | |
| 27 | PSLVMPPSPSPLV | NC_002645_Human_coronavirus | 229E |
| | | _229E_Translation_RF-3_42 | |
| 28 | QAAAAMYKEARAVN | NCBI ref. seq. NP_073549.1 | 229E |
| 29 | QTSQALQTVATALNK | NCBI ref. seq. NP_073549.1 | 229E |
| 30 | SEISANGCSTIAQA | NCBI ref. seq. NP_073549.1 | 229E |
| 31 | SNFNTLFATTIPN | NCBI ref. seq. NP_073549.1 | 229E |
| 32 | TIQGPPGSGKS | NCBI ref. seq. NP_073549.1 | 229E |
| 33 | TNVPLQVGFSNG | NCBI ref. seq. NP_073549.1 | 229E |
| 34 | VGGTIQIL | NCBI ref. seq. NP_073549.1 | 229E |
| 35 | VLFSATAVKTGGK | NCBI ref. seq. NP_073549.1 | 229E |
| 36 | VLNNGFGGKQI | NCBI ref. seq. NP_073549.1 | 229E |
| 37 | VTSGLGTVDADY | NCBI ref. seq. NP_073549.1 | 229E |

229E = common cold coronavirus 229E

To identify SEQ ID NOs: 1 to 37, HepG2 cells were infected with 229E, harvested, lysed and MHC class I peptides were extracted to generate peptide sample mixture for mass spectrometry analysis. An ultra-high pressure liquid chromatography (UPLC) system coupled with an Eclipse triploid mass spectrometer was used for peptide separation and identification. Additionally, a control sample (mock infection) was collected and processed following the same procedure as for samples from infected cells. One collision mode (CID) were applied in analysis by mass spectrometry. A data dependent acquisition (DDA) method was implemented for real time sample acquisition. The generated datasets were processed using SEQUEST HT software for the database searching against 229E protein sequence in 1) positive sense translations, 2) negative sense translations, and 3) IPI human database. Full experimental details are set out in Example 1.

The peptide of the invention is thus capable of binding to a MHC class I molecule. The peptide may be derived from the immunoproteosome processing of the viral proteome inside an infected cell. The peptide may be a peptide that is expressed on the surface of one or more coronaviruses, or intracellularly within one or more coronaviruses. The peptide may, for example, be a structural peptide. The peptide may, for example, be a functional peptide, such as a peptide involved in the metabolism or replication of one or more coronaviruses. The peptide may, for example, be an internal peptide. The coronavirus may, for example, be a common cold coronavirus such as 229E, NL63, OC43, and HKU1. Preferably, the coronavirus is 229E.

Preferably, the peptide is conserved between two or more different coronaviruses or coronavirus serotypes. For example, the peptide may be conserved between 229E and one or more NL63, OC43 and HKU1. For instance, the peptide may be conserved between 229E and NL63; 229E and OC43; 229E and HKU1; 229E, NL63 and OC43; 229E, NL63 and HKU1; 229E, OC43 and HKU1; or 229E, NL63, OC43 and HKU1. A peptide is conserved between two or more different coronaviruses or coronavirus serotypes if each of the two or more different coronaviruses or coronavirus serotypes encodes a sequence that is 50% or more (such as 60%, 70%, 75%, 80%, 90%, 95%, 98% or 99%) homologous to the peptide.

The peptide may comprise only one sequence selected from SEQ ID NOs: 1 to 37 and a variant of any thereof. Alternatively, the peptide may comprise two or more, such as three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, or 37 such sequences, in any combination.

The peptide may comprise multiple copies of any sequence selected from SEQ ID NOs: 1 to 37 and a variant of any thereof. For example, the peptide may comprise two or more, three or more, four or more, five or more, six or more, seven or more, eight or more, nine or more, ten or more copies of any sequence selected from SEQ ID NOs: 1 to 37 and a variant of any thereof. In such peptides, the multiple copies may be joined directly to one another, or may be joined by one or more linking amino acids, such as from 2 to about 20 amino acids or about 3 to about 10 amino acids. In the peptide, the linking amino acids typically do not comprise the exact amino acid sequence that links the sequences of two or more of SEQ ID NOs: 1 to 37 in nature.

The peptide may contain any number of amino acids, i.e. be of any length. For example, the peptide may be about 8, about 9, about 10, about 15, about 20, about 25, about 30, about 35, about 40, about 45, about 50, about 55, about 60, about 65, about 70, about 75, about 80, about 85, about 90, about 95 or about 100 amino acids in length. For example, the peptide may be about 8 to about 100, about 9 to about 95, about 10 to about 90, about 15 to about 85, about 20 to about 80, about 25 to about 75, about 30 to about 70, about 35 to about 65, about 40 to about 60, about 45 to about 55, or about 50 amino acids in length. Typically, the peptide is about 8 to about 30, 35 or 40 amino acids in length, such as about 9 to about 29, about 10 to about 28, about 11 to about 27, about 12 to about 26, about 13 to about 25, about 13 to about 24, about 14 to about 23, about 15 to about 22, about 16 to about 21, about 17 to about 20, or about 18 to about 29 amino acids in length. The peptide may consist of, or consist essentially of, the amino acid sequence of one of SEQ ID NOs: 1 to 37.

As well as a sequence selected from SEQ ID NOs: 1 to 37 or a variant of any thereof, the peptide may comprise one or more CD8+ T cell epitopes, one or more CD4+ T cell epitopes and/or one or more B cell epitopes. For example, the peptide may comprise two or more, such as three or more, four or more, five or more, ten or more, fifteen or more, or twenty or more CD8+ T cell epitopes. The peptide may comprise two or more, such as three or more, four or more, five or more, ten or more, fifteen or more, or twenty or more CD4+ T cell epitopes. The peptide may comprise two or more, such as three or more, four or more, five or more, ten or more, fifteen or more, or twenty or more B cell epitopes.

The CD8+ T cell epitope is preferably a CD8+ T cell epitope that does not comprise any one of SEQ ID NOs: 1 to 37 or a variant of any thereof. The CD8+ T cell epitope may, for example, be a coronavirus CD8+ epitope, i.e. a peptide that is expressed by one or more coronaviruses and that is that is capable of (i) presentation by a class I MHC molecule and (ii) recognition by a T cell receptor (TCR) present on a CD8+ T cell. Preferably, the coronavirus is a common cold coronavirus. More preferably, the coronavirus is 229E. The CD8+ T cell epitope may alternatively be a CD8+ T cell epitope that is not expressed by one or more coronaviruses, such as 229E.

The CD4+ T cell epitope may, for example, be a coronavirus CD4+ epitope, i.e. a peptide that is expressed by one or more coronaviruses and that is that is capable of (i) presentation by a class II MHC molecule and (ii) recognition by a T cell receptor (TCR) present on a CD4+ T cell. Preferably, the coronavirus is a common cold coronavirus. More preferably, the coronavirus is 229E. The CD4+ T cell epitope may alternatively be a CD4+ T cell epitope that is not expressed by one or more coronaviruses, such as 229E.

The B cell epitope may, for example, be a coronavirus B cell epitope, i.e. a peptide that is expressed by one or more coronaviruses and that is that is capable of recognition by a B cell receptor (BCR) present on a B cell. Preferably, the coronavirus is a common cold coronavirus. More preferably, the coronavirus is 229E. The B cell epitope may alternatively be a B cell epitope that is not expressed by one or more coronaviruses, such as 229E.

SEQ ID NOs: 20 (LIAGKLLPPV) and 27 (PSLVMPPSPSPLV) are so-called “reverse peptides” from 229E. Reverse peptides from coronaviruses are described in detail in Greek patent application no. 20210100099 filed on 16 Feb. 2021. In brief, a reverse peptide is an amino acid sequence that is comprised in a polypeptide that is encoded by an open reading frame (ORF) encoded by at least part of the genome of a virus in the opposite sense to positive sense RNA capable of translation. A reading frame is a grouping of three successive nucleotides in a nucleic acid sequence, such as an RNA sequence, that constitutes the codons for the amino acids encoded by the nucleic acid sequence. An ORF (open reading frame) is the part of a reading frame that can be translated. An ORF is a continuous stretch of codons that contain a start codon and a stop codon. Within the ORF, an initiation codon (e.g. ATG) may serve as an initiation site for translation of the RNA into protein.

For a coronavirus reverse peptide, the virus whose genome encodes the ORF virus is a coronavirus. Coronaviruses are positive-sense ssRNA viruses. In a positive-sense ssRNA virus, the viral genomic RNA is the same sense as the mRNA and can be translated without intervening transcription i.e. the genomic RNA doubles as mRNA. Here, the ORF is encoded in the opposite sense to the viral genomic RNA and, therefore, the mRNA. Accordingly, the ORF is negative sense. That is, the ORF requires the genomic RNA to be transcribed before it is capable of translation.

The positive sense genome of certain coronaviruses, such as 229E, comprise a gene encoding the polyprotein pplab (NCBI protein ID NP_073549.1. The inventors have discovered that the pplab gene also encodes, in the opposite sense to positive sense RNA capable of translation, two ORFs giving rise to polypeptides that comprise SEQ ID NOs: 20 (LIAGKLLPPV) and 27 (PSLVMPPSPSPLV). In other words, the inventors have discovered that the negative sense (i.e. reverse complement of) of the pplab gene also encodes an ORF encoding a polypeptide that comprise SEQ ID NO: 20 (LIAGKLLPPV) and an ORF encoding a polypeptide that comprises SEQ ID NO: 27 (PSLVMPPSPSPLV). In other words, at least part of the pplab gene may be transcribed to give complementary mRNA that comprises the ORF encoding a polypeptide comprising SEQ ID NOs: 20 (LIAGKLLPPV). The ORF encoding the polypeptide giving rise SEQ ID NO: 20 (LIAGKLLPPV) may therefore be encoded by at least part of the pplab gene in the opposite sense to positive sense RNA capable of translation. At least part of the pplab gene may be transcribed to give complementary mRNA that comprises the ORF encoding a polypeptide comprising SEQ ID NO: 27 (PSLVMPPSPSPLV). The ORF encoding the polypeptide giving rise SEQ ID NO: 27 (PSLVMPPSPSPLV) may therefore be encoded by at least part of the pplab gene in the opposite sense to positive sense RNA capable of translation.

Any peptide of the invention may be chemically derived from a polypeptide antigen, for example by proteolytic cleavage. More typically, the peptide may be synthesised using methods well known in the art. The term “peptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2 may be modified to —NH(Me) or —N(Me)₂).

The term “peptide” also includes peptide variants that increase or decrease the half-life of the peptide in vivo. Examples of analogues capable of increasing the half-life of peptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

### Variants

As set out above, the peptide may comprise a variant of any one of SEQ ID NOs: 1 to 37. The peptide may, for example, comprise a variant of two or more, such as three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, or 37 of SEQ ID NOs: 1 to 37.

A variant of any one of SEQ ID NOs: 1 to 37 may be a sequence that differs from the corresponding sequence selected from SEQ ID NOs: 1 to 37 by no more than five (such as no more than four, no more than three, no more than two, or no more than one) amino acid(s). Each of the no more than five amino acid differences may be an amino acid substitution, deletion or insertion relative to the corresponding sequence selected from SEQ ID NOs: 1 to 37. The amino acid substitution may, for example, be a conservative amino acid substitution.

Preferably, a variant of any one of SEQ ID NOs: 1 to 37 is a sequence that differs from the respective one of SEQ ID NOs: 1 to 37 by no more than one amino acid. That is, the variant may differ from the corresponding sequence selected from SEQ ID NOs: 1 to 37 by a single amino acid. The single amino acid difference may, for example, be a single amino acid substitution, deletion or insertion relative to the corresponding sequence selected from SEQ ID NOs: 1 to 37. The single amino acid substitution may, for example, be a conservative amino acid substitution.

Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 2 below. Where amino acids have similar polarity, this can also be determined by reference to the hydropathy scale for amino acid side chains in Table 3.

TABLE 2
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, |
| | | | charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, hydrophobic |

TABLE 3
Hydropathy scale

| Side chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

### Polynucleotides

The invention provides a polynucleotide encoding a peptide of the invention. Peptides of the invention are described in detail above. Any of the aspects described above in connection with the peptide of the invention may apply to the polynucleotide of the invention.

The polynucleotide may comprise RNA. The polynucleotide may comprise DNA. The polynucleotide may comprise both RNA and DNA. Preferably, the polynucleotide comprises or consists of RNA. More preferably, the polynucleotide comprises or consists of mRNA.

### Pharmaceutical Composition

The present invention provides a pharmaceutical composition comprising the peptide of the invention or the polynucleotide of the invention. Peptides of the invention and polynucleotides of the invention are described in detail above. Any of the aspects described above in connection with the peptide of the invention or the polynucleotide of the invention may apply to the pharmaceutical composition of the invention.

The pharmaceutical composition may, for example, be a prophylactic composition, such as a vaccine. The pharmaceutical composition may, for example, be a therapeutic composition. The pharmaceutical composition may have prophylactic effect, therapeutic effect, or both prophylactic and therapeutic effect. In other words, the pharmaceutical composition may have utility in preventing coronavirus infection, treating coronavirus infection, or both preventing and treating coronavirus infection.

The pharmaceutical composition has a number of benefits. Firstly, the pharmaceutical composition is capable of stimulating an immune response against a common cold coronavirus. Preferably, the immune response is a cellular immune response (e.g. a CD8+ T cell response). CD8+ cytotoxic T lymphocytes (CTLs) mediate viral clearance via their cytotoxic activity against infected cells. Stimulating cellular immunity may therefore provide a beneficial defence against common cold coronavirus infection.

Secondly, the peptides identified by the present inventors may be conserved between different coronaviruses and may be presented by MHC molecules on cells infected with one or more of those viruses. For example, the peptides identified by the present inventors may be conserved between different common cold coronaviruses, such as 229E and one or more of NL63, OC43, and HKU1. Inclusion of conserved peptides in the pharmaceutical composition may confer protective capability against multiple common cold, i.e. confer cross-protection. 100% homology between viruses is not required for cross-protection to be conferred. Rather, cross-protection may arise following immunisation with a sequence that is, for example, about 50% or more (such as 60%, 70%, 75%, 80%, 90%, 95%, 98% or 99%) homologous to a CD8+ T cell epitope expressed in a cell infected with a different virus, if certain residues are retained in the correct position. A pharmaceutical composition comprising one or more peptides comprising a sequence selected from SEQ ID NOs: 1 to 37 or a variant of any thereof may therefore be capable of providing cross-protection against a variety of existing coronaviruses (such as common cold coronaviruses) over and above 229E. Inclusion of one or more conserved peptides in the pharmaceutical composition may also confer protective capability against emerging coronavirus strains associated with evolution of the coronavirus genome. In this way, a single coronavirus pharmaceutical composition can be used to confer protection against a variety of different coronaviruses. This provides a cost-effective means of controlling the spread of coronavirus infection.

Thirdly, different peptides identified by the present inventors may be capable of binding to different HLA supertypes. Inclusion of multiple peptides each capable of binding to a different HLA supertype (or corresponding polynucleotides) results in a pharmaceutical composition that is effective in individuals having different HLA types. In this way, a single pharmaceutical composition can be used to confer protection in a large proportion of the human population.

Fourthly, the peptide or polynucleotide comprised in the pharmaceutical composition of the invention may be attached to a nanoparticle, for example a gold nanoparticle. As described in more detail below, attachment to a nanoparticle reduces or eliminates the need to include an adjuvant in the pharmaceutical composition. Attachment to a nanoparticle also reduces or eliminates the need to include a virus in the pharmaceutical composition. Thus, the pharmaceutical composition of the invention is less likely to cause adverse clinical effects upon administration to an individual.

### Peptide Compositions

The pharmaceutical composition may comprise one or more peptides, such as about one to about 50, about 2 to about 40, about 3 to about 30, about 4 to about 25, about 5 to about 20, about 6 to about 15, about 7, about 8, about 9 or about 10 peptides. Each of the one or more peptides may, for example, be an immunogenic peptide of the invention. Alternatively, the pharmaceutical composition may contain a mixture of (i) peptides of the invention and (ii) other peptides such as other immunogenic peptides.

In one aspect, the pharmaceutical comprises two or more peptides. Each of the two or more peptides may be a peptide of the invention. The pharmaceutical composition may, for example, comprise two or more different peptides of the invention. In other words, the pharmaceutical composition may comprise two or more peptides that each comprise a different sequence selected from SEQ ID NOs: 1 to 37 or a variant of any thereof. The pharmaceutical composition may comprise about one to about 50, about 2 to about 40, about 3 to about 30, about 4 to about 25, about 5 to about 20, about 6 to about 15, about 7, about 8, about 9 or about 10 such different peptides. In some aspects, each of the different peptides may interact with a different HLA subtype.

The pharmaceutical composition may comprise (i) one or more (such as about 2 to about 40, about 3 to about 30, about 4 to about 25, about 5 to about 20, about 6 to about 15, about 7, about 8, about 9 or about 10) peptides of the invention, and (ii) one or more other peptides (i.e. peptides that do not comprise a sequence selected from SEQ ID NOs: 1 to 37 or a variant of any thereof). Preferably, the one or more other peptides are immunogenic peptide. The one or more other peptides may comprise one or more epitopes, such as about 2 to 40, about 3 to about 30, about 4 to about 25, about 5 to about 20, about 6 to about 15, about 7, about 8, about 9 or about 10 epitopes. Each of the one or more epitopes may, for example, be a B cell epitope, a CD4+ T cell epitope and/or CD8+ T cell epitope. The CD4+ T cell epitope may, for example, be a peptide that is expressed by one or more coronaviruses and that is capable of (i) presentation by a class II MHC molecule and (ii) recognition by a T cell receptor (TCR) present on a CD4+ T cell. Alternatively, the CD4+ T cell epitope may be an CD4+ T cell epitope that is not expressed by one or more coronaviruses. The CD8+ T cell epitope may, for example, be a peptide that is expressed by one or more coronaviruses and that is capable of (i) presentation by a class I MHC molecule and (ii) recognition by a T cell receptor (TCR) present on a CD8+ T cell. The CD8+ T cell epitope may be an CD8+ T cell epitope that is not expressed by one or more coronaviruses. The B cell epitope may, for example, be a peptide that is expressed by one or more coronaviruses and that is capable of recognition by a B cell receptor (BCR) present on a B cell. The B cell epitope may be a B cell epitope that is not expressed by one or more coronaviruses.

Many B cell epitopes, CD4+ T cell epitopes and CD8+ T cell epitopes (such as B cell epitopes, CD4+ T cell epitopes and CD8+ T cell epitopes from coronaviruses) are known in the art. Methods for identifying B cell epitopes, CD4+ T cell epitopes and CD8+ T cell epitopes are known in the art. Epitope mapping methods include X-ray co-crystallography, array-based oligo-peptide scanning (sometimes called overlapping peptide scan or pepscan analysis), site-directed mutagenesis, high throughput mutagenesis mapping, hydrogen-deuterium exchange, crosslinking coupled mass spectrometry, phage display and limited proteolysis. MHC motif prediction methodologies may also be used. CD8+ T cell epitopes presented by coronavirus-infected cells can be identified in order to directly identify CD8+ T cell epitopes for inclusion in the pharmaceutical composition, as described above. B cell epitopes may be identified using epitope mapping methods. These methods include structural approaches, wherein the known or modelled structure of a protein is used in an algorithm based approach to predict surface epitopes, and functional approaches, wherein the binding of whole proteins, protein fragments or peptides to an antibody can be quantitated e.g. using an Enzyme-Linked Immunosorbent Assay (ELISA). Competition mapping, antigen modification or protein fragmentation methods may also be used.

The pharmaceutical composition may comprise at least one peptide of the invention that interacts with at least two different HLA supertypes. This may allow the pharmaceutical composition to elicit an immune response in a greater proportion of individuals to which the pharmaceutical composition is administered. This is because the pharmaceutical composition should be capable of eliciting an immune response (such as a T cell response, e.g. a CD8+ T cell response) in all individuals of an HLA supertype that interacts with the peptide. The pharmaceutical composition may, for example, comprise at least one, at least two, at least three, at least four, at least five, at least ten, at least fifteen, or at least twenty peptides of the invention that each interact with at least two different HLA supertypes. Each peptide may interact with at least two, at least three, at least four, at least five, at least six, at least 7, at least 8, at least 9 or at least 10 different HLA supertypes. Each peptide may interact with two or more of A1, A10, A11, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, and C9, or any other HLA supertype known in the art, in any combination.

The pharmaceutical composition may comprise at least two peptides of the invention that each interact with a different HLA supertype. For example, the pharmaceutical composition may comprise two or more peptides that (i) each comprise a different sequence selected from SEQ ID NOs: 1 to 37 or a variant of any thereof, and (ii) each interact with a different HLA supertype. Including two or more such peptides in the pharmaceutical composition allows the pharmaceutical composition to elicit an immune response in a greater proportion of individuals to which the pharmaceutical composition is administered. This is because the pharmaceutical composition should be capable of eliciting an immune response (such as a T cell response) in all individuals of an HLA supertype that interacts with one of the sequences comprised in the two or more peptides. Each peptide may interact with A1, A10, A11, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, or C9, or any other HLA supertype know in the art. Any combination of peptides is possible.

### Polynucleotide Composition

The pharmaceutical composition may comprise one or more polynucleotides, such as about one to about 50, about 2 to about 40, about 3 to about 30, about 4 to about 25, about 5 to about 20, about 6 to about 15, about 7, about 8, about 9 or about 10 polynucleotides. Each of the one or more polynucleotides may, for example, be a polynucleotide of the invention. Alternatively, the pharmaceutical composition may contain a mixture of (i) polynucleotides of the invention and (ii) other polynucleotides.

Each polynucleotide may comprise RNA, such as mRNA. Each polynucleotide may comprise DNA. Each polynucleotide may comprise RNA and DNA. Preferably, each polynucleotide comprises or consists of RNA. More preferably, each polynucleotide comprises or consists of mRNA.

In one aspect, the pharmaceutical composition comprises two or more polynucleotides. Each of the two or more polynucleotides may be a polynucleotide of the invention. The pharmaceutical composition may, for example, comprise two or more polynucleotides each encoding a different peptide of the invention. In other words, the pharmaceutical composition may comprise two or more polynucleotides that each encode a different peptide comprising a sequence selected from SEQ ID NOs: 1 to 37 or a variant of any thereof. The pharmaceutical composition may comprise about one to about 50, about 2 to about 40, about 3 to about 30, about 4 to about 25, about 5 to about 20, about 6 to about 15, about 7, about 8, about 9 or about 10 polynucleotides each encoding a different peptide comprising a sequence selected from SEQ ID NOs: 1 to 37 or a variant of any thereof. In some aspects, each of the different peptides may interact with a different HLA subtype.

The pharmaceutical composition may comprise one or more (such as about 2 to about 40, about 3 to about 30, about 4 to about 25, about 5 to about 20, about 6 to about 15, about 7, about 8, about 9 or about 10) polynucleotides each encoding a peptide of the invention, and (ii) one or more other polynucleotides (i.e. polynucleotides that do not encode a peptide that comprises a sequence selected from SEQ ID NOs: 1 to 37 or a variant of any thereof). The other polynucleotides may encode one or more epitopes, such as about 2 to 40, about 3 to about 30, about 4 to about 25, about 5 to about 20, about 6 to about 15, about 7, about 8, about 9 or about 10 epitopes. The epitope may, for example, be B cell epitope, a CD4+ T cell epitope and/or CD8+ T cell epitope, as described above in connection with peptide vaccines.

The pharmaceutical composition may comprise at least one polynucleotide that encodes an peptide that interacts with at least two different HLA supertypes. This allows the pharmaceutical composition to elicit an immune response (such as a T cell response, e.g. a CD8+ T cell response) in a greater proportion of individuals to which the pharmaceutical composition is administered. This is because the pharmaceutical composition should be capable of eliciting an immune response (such as a T cell response, e.g. a CD8+ T cell response) in all individuals of an HLA supertype that interacts with the epitope. The pharmaceutical composition may, for example, comprise at least one, at least two, at least three, at least four, at least five, at least ten, at least fifteen, or at least twenty polynucleotides that each encode a peptide that comprises a sequence selected from SEQ ID NOs: 1 to 37 or a variant of any thereof and interacts with at least two different HLA supertypes. Each peptide may interact with at least two, at least three, at least four, at least five, at least six, at least 7, at least 8, at least 9 or at least 10 different HLA supertypes. Each peptide may interact with two or more of A1, A10, A11, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, and C9, or any other HLA supertype known in the art, in any combination.

The pharmaceutical composition may comprise at least two polynucleotides that each encode a peptide that interacts with a different HLA supertype. For example, the pharmaceutical composition may comprise two or more polynucleotides that each encode a peptide that (i) comprises a different sequence selected from SEQ ID NOs: 1 to 37 or a variant of any thereof, and (ii) interacts with a different HLA supertype. Including two or more such polynucleotides in the pharmaceutical composition allows the pharmaceutical composition to elicit an immune response (such as a T cell response, e.g. a CD8+ T cell response) in a greater proportion of individuals to which the composition is administered. This is because the pharmaceutical composition should be capable of eliciting an immune response (such as a T cell response, e.g. a CD8+ T cell response) in all individuals of an HLA supertype that interacts with one of the epitopes encoded by the two or more polynucleotides. Each peptide may interact with A1, A10, A11, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, or C9, or any other HLA supertype know in the art. Any combination of peptides is possible.

### Nanoparticles

Any peptide or polynucleotide comprised in the pharmaceutical composition of the invention may be attached to a nanoparticle. Attachment may, for example, be via a linker. Attachment to a nanoparticle, for example a gold nanoparticle, is beneficial.

Attachment of the peptide or polynucleotide to a nanoparticle (such as a gold nanoparticle) reduces or eliminates the need to include a virus or an adjuvant in the pharmaceutical composition. The nanoparticles may contain immune “danger signals” that help to effectively induce an immune response to the peptides or polynucleotides. The nanoparticles may induce dendritic cell (DC) activation and maturation, required for a robust immune response. The nanoparticles may contain non-self components that improve uptake of the nanoparticles and thus the peptides or polynucleotides by cells, such as antigen presenting cells. Attachment of a peptide or polynucleotide to a nanoparticle may therefore enhance the ability of antigen presenting cells to stimulate virus-specific T and/or B cells. Attachment to a nanoparticle also facilitates delivery of the pharmaceutical compositions via the subcutaneous, intradermal, transdermal and oral/buccal routes, providing flexibility in administration.

Nanoparticles are particles between 1 and 100 nanometers (nm) in size which can be used as a substrate for immobilising ligands. In the pharmaceutical compositions of the invention, the nanoparticle may have a mean diameter of 1 to 100, 20 to 90, 30 to 80, 40 to 70 or 50 to 60 nm. Preferably, the nanoparticle has a mean diameter of 20 to 40 nm. A mean diameter of 20 to 40 nm facilitates uptake of the nanoparticle to the cytosol. The mean diameter can be measured using techniques well known in the art such as transmission electron microscopy.

Nanoparticles suitable for the delivery of antigen, such as an immunogenic peptide, are known in the art. Methods for the production of such nanoparticles are also known. Nanoparticles suitable for the delivery of polynucleotides, and methods for their production are also known in the art.

The nanoparticle may, for example, be a polymeric nanoparticle, an inorganic nanoparticle, a liposome, an immune stimulating complex (ISCOM), a virus-like particle (VLP), or a self-assembling protein. The nanoparticle is preferably a calcium phosphate nanoparticle, a silicon nanoparticle or a gold nanoparticle.

The nanoparticle may be a polymeric nanoparticle. The polymeric nanoparticle may comprise one or more synthetic polymers, such as poly(d,l-lactide-co-glycolide) (PLG), poly(d,l-lactic-coglycolic acid) (PLGA), poly(g-glutamic acid) (g-PGA)m poly(ethylene glycol) (PEG), or polystyrene. The polymeric nanoparticle may comprise one or more natural polymers such as a polysaccharide, for example pullulan, alginate, inulin, and chitosan. The use of a polymeric nanoparticle may be advantageous due to the properties of the polymers that may be included in the nanoparticle. For instance, the natural and synthetic polymers recited above may have good biocompatibility and biodegradability, a non-toxic nature and/or the ability to be manipulated into desired shapes and sizes. The polymeric nanoparticle may form a hydrogel nanoparticle. Hydrogel nanoparticles are a type of nano-sized hydrophilic three-dimensional polymer network. Hydrogel nanoparticles have favourable properties including flexible mesh size, large surface area for multivalent conjugation, high water content, and high loading capacity for antigens. Polymers such as Poly(L-lactic acid) (PLA), PLGA, PEG, and polysaccharides are particularly suitable for forming hydrogel nanoparticles.

The nanoparticle may be an inorganic nanoparticle. Typically, inorganic nanoparticles have a rigid structure and are non-biodegradable. However, the inorganic nanoparticle may be biodegradable. The inorganic nanoparticle may comprise a shell in which an antigen may be encapsulated. The inorganic nanoparticle may comprise a core to which an antigen may be covalently attached. The core may comprise a metal. For example, the core may comprise gold (Au), silver (Ag) or copper (Cu) atoms. The core may be formed of more than one type of atom. For instance, the core may comprise an alloy, such as an alloy of Au/Ag, Au/Cu, Au/Ag/Cu, Au/Pt, Au/Pd or Au/Ag/Cu/Pd. The core may comprise calcium phosphate (CaP). The core may comprise a semiconductor material, for example cadmium selenide. The nanoparticle may be a gold nanoparticle, such as a gold glyco-nanoparticle.

Other exemplary inorganic nanoparticles include carbon nanoparticles and silica-based nanoparticles. Carbon nanoparticles have good biocompatibility and can be synthesized into nanotubes and mesoporous spheres. Silica-based nanoparticles (SiNPs) are biocompatible and can be prepared with tunable structural parameters to suit their therapeutic application.

The nanoparticle may be a silicon nanoparticle, such as an elemental silicon nanoparticle. The nanoparticle may be mesoporous or have a honeycomb pore structure. Preferably, the nanoparticle is an elemental silicon particle having a honeycomb pore structure. Such nanoparticles are known in the art and offer tunable and controlled drug loading, targeting and release that can be tailored to almost any load, route of administration, target or release profile. For example, such nanoparticles may increase the bioavailability of their load, and/or improve the intestinal permeability and absorption of orally administered actives. The nanoparticles may have an exceptionally high loading capacity due to their porous structure and large surface area. The nanoparticles may release their load over days, weeks or months, depending on their physical properties. Since silicon is a naturally occurring element of the human body, the nanoparticles may elicit no response from the immune system. This is advantageous to the in vivo safety of the nanoparticles.

Any of the SiNPs described above may be biodegradable or non-biodegradable. A biodegradable SiNP may dissolve to orthosilic acid, the bioavailable form of silicon. Orthosilic acid has been shown to be beneficial for the health of bones, connective tissue, hair, and skin.

The nanoparticle may be a liposome. Liposomes are typically formed from biodegradable, non-toxic phospholipids and comprise a self-assembling phospholipid bilayer shell with an aqueous core. A liposome may be an unilamellar vesicle comprising a single phospholipid bilayer, or a multilameller vesicle that comprises several concentric phospholipid shells separated by layers of water. As a consequence, liposomes can be tailored to incorporate either hydrophilic molecules into the aqueous core or hydrophobic molecules within the phospholipid bilayers. Liposomes may encapsulate antigen within the core for delivery. Liposomes may incorporate viral envelope glycoproteins to the shell to form virosomes. A number of liposome-based products are established in the art and are approved for human use.

The nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine) and saponin (such as Quil A from the tree Quillaia saponaria). ISCOMs have traditionally been used to entrap viral envelope proteins, such as envelope proteins from herpes simplex virus type 1, hepatitis B, or influenza virus.

The nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

The nanoparticle may be a self-assembling protein. For instance, the nanoparticle may comprise ferritin. Ferritin is a protein that can self-assemble into nearly-spherical 10 nm structures. The nanoparticle may comprise major vault protein (MVP). Ninety-six units of MVP can self-assemble into a barrel-shaped vault nanoparticle, with a size of approximately 40 nm wide and 70 nm long.

The nanoparticle may be a calcium phosphate (CaP) nanoparticle. CaP nanoparticles may comprise a core comprising one or more (such as two or more, 10 or more, 20 or more, 50 or more, 100 or more, 200 or more, or 500 or more) molecules of CaP. CaP nanoparticles and methods for their production are known in the art. For instance, a stable nano-suspension of CAP nanoparticles may be generated by mixing inorganic salt solutions of calcium and phosphates in pre-determined ratios under constant mixing.

The CaP nanoparticle may have an average particle size of about 80 to about 100 nm, such as about 82 to about 98 nm, about 84 to about 96 nm, about 86 to about 94 nm, or about 88 to about 92 nm. This particle size may produce a better performance in terms of immune cell uptake and immune response than other, larger particle sizes. The particle size may be stable (i.e. show no significant change), for instance when measured over a period of 1 month, 2 months, 3 months, 6 months, 12 months, 18 months, 24 months, 36 months or 48 months.

CaP nanoparticles can be co-formulated with one or multiple antigens either adsorbed on the surface of the nanoparticle or co-precipitated with CaP during particle synthesis. For example, a peptide, such as an immunogenic peptide, may be attached to the CaP nanoparticle by dissolving the peptide in DMSO (for example at a concentration of about 10 mg/ml), adding to a suspension of CaP nanoparticles together with N-acetyl-glucosamine (GlcNAc) (for example at 0.093 mol/L and ultra-pure water, and mixing at room temperature for a period of about 4 hours (for example, 1 hour, 2 hours, 3 hours, 5 hours, 6 hours, 7 hours, 8 hours, 9 hours or 10 hours).

The pharmaceutical composition may comprise about 0.15 to about 0.8%, such as 0.2 to about 0.75%, 0.25 to about 0.7%, 0.3 to about 0.6%, 0.35 to about 0.65%, 0.4 to about 0.6%, or 0.45 to about 0.55%, CaP nanoparticles. Preferably the pharmaceutical composition comprises about 0.3% CaP nanoparticles.

CaP nanoparticles have a high degree of biocompatibility due to their chemical similarity to human hard tissues such as bone and teeth. Advantageously, therefore, CaP nanoparticles are non-toxic when used for therapeutic applications. CaP nanoparticles are safe for administration via intramuscular, subcutaneous, oral, or inhalation routes. CaP nanoparticles are also simple to synthesise commercially. Furthermore, CaP nanoparticles may be associated with slow release of antigen, which may enhance the induction of an immune response to a peptide attached to the nanoparticle. CaP nanoparticles may be used both as an adjuvant, and as a drug delivery vehicle.

The nanoparticle may be a gold nanoparticle, such as a gold glyconanoparticle. Gold nanoparticles are known in the art and are described in particular in WO 2002/32404, WO 2006/037979, WO 2007/122388, WO 2007/015105 and WO 2013/034726. The gold nanoparticle attached to each peptide may be a gold nanoparticle described in any of WO 2002/32404, WO 2006/037979, WO 2007/122388, WO 2007/015105 and WO 2013/034726.

Gold nanoparticles comprise a core comprising a gold (Au) atom. The core may further comprise one or more Fe, Cu or Gd atoms. The core may be formed from a gold alloy, such as Au/Fe, Au/Cu, Au/Gd, Au/Fe/Cu, Au/Fe/Gd or Au/Fe/Cu/Gd. The total number of atoms in the core may be 100 to 500 atoms, such as 150 to 450, 200 to 400 or 250 to 350 atoms. The gold nanoparticle may have a mean diameter of 1 to 100, 20 to 90, 30 to 80, 40 to 70 or 50 to 60 nm. Preferably, the gold nanoparticle has a mean diameter of 20 to 40 nm.

The nanoparticle may comprise a surface coated with alpha-galactose and/or beta-GlcNHAc. For instance, the nanoparticle may comprise a surface passivated with alpha-galactose and/or beta-GlcNHAc. In this case, the nanoparticle may, for example, be a nanoparticle which comprises a core including metal and/or semiconductor atoms. For instance, the nanoparticle may be a gold nanoparticle. Beta-GlcNHAc is a bacterial pathogen-associated-molecular pattern (PAMP), which is capable of activating antigen-presenting cells. In this way, a nanoparticle comprising a surface coated or passivated with Beta-GlcNHAc may non-specifically stimulate an immune response. Attachment of an immunogenic peptide to such a nanoparticle may therefore improve the immune response elicited by administration of the pharmaceutical composition of the invention to an individual.

One or more ligands other than the peptide or polynucleotide may be linked to the nanoparticle, which may be any of the types of nanoparticle described above. The ligands may form a “corona”, a layer or coating which may partially or completely cover the surface of the core. The corona may be considered to be an organic layer that surrounds or partially surrounds the nanoparticle core. The corona may provide or participate in passivating the core of the nanoparticle. Thus, in certain cases the corona may be a sufficiently complete coating layer to stabilise the core. The corona may facilitate solubility, such as water solubility, of the nanoparticles of the present invention.

The nanoparticle may comprise at least 10, at least 20, at least 30, at least 40 or at least 50 ligands. The ligands may include one or more peptides, protein domains, nucleic acid molecules, lipidic groups, carbohydrate groups, anionic groups, or cationic groups, glycolipids and/or glycoproteins. The carbohydrate group may be a polysaccharide, an oligosaccharide or a monosaccharide group (e.g. glucose). One or more of the ligands may be a non-self component, that renders the nanoparticle more likely to be taken up by antigen presenting cells due to its similarity to a pathogenic component. For instance, one or more ligands may comprise a carbohydrate moiety (such as a bacterial carbohydrate moiety), a surfactant moiety and/or a glutathione moiety. Exemplary ligands include glucose, N-acetylglucosamine (GlcNAc), glutathione, 2′-thioethyl-β-D-glucopyranoside and 2′-thioethyl-D-glucopyranoside. Preferred ligands include glycoconjugates, which form glyconanoparticles

Linkage of the ligands to the core may be facilitated by a linker. The linker may comprise a thiol group, an alkyl group, a glycol group or a peptide group. For instance, the linker may comprise C2-C15 alkyl and/or C2-C15 glycol. The linker may comprise a sulphur-containing group, amino-containing group, phosphate-containing group or oxygen-containing group that is capable of covalent attachment to the core. Alternatively, the ligands may be directly linked to the core, for example via a sulphur-containing group, amino-containing group, phosphate-containing group or oxygen-containing group comprised in the ligand.

### Attachment to Nanoparticles

Typically, the immunogenic peptide or polynucleotide may be attached to the core of the nanoparticle, but attachment to the corona or a ligand may also be possible. An immunogenic peptide may be attached at its N-terminus to the nanoparticle.

The peptide or polynucleotide may be directly attached to the nanoparticle, for example by covalent bonding of an atom in a sulphur-containing group, amino-containing group, phosphate-containing group or oxygen-containing group in the peptide or polynucleotide to an atom in the nanoparticle or its core.

A linker may be used to link the peptide or polynucleotide to the nanoparticle. The linker may comprise a sulphur-containing group, amino-containing group, phosphate-containing group or oxygen-containing group that is capable of covalent attachment to an atom in the core. For example, the linker may comprise a thiol group, an alkyl group, a glycol group or a peptide group.

The linker may comprise a peptide portion and a non-peptide portion. The peptide portion may comprise the sequence X₁X₂Z₁, wherein X₁ is an amino acid selected from A and G; X₂ is an amino acid selected from A and G; and Z₁ is an amino acid selected from Y and F. The peptide portion may comprise the sequence AAY or FLAAY (SEQ ID NO: 38). The peptide portion of the linker may be linked to the N-terminus of the peptide. The non-peptide portion of the linker may comprise a C2-C15 alkyl and/a C2-C15 glycol, for example a thioethyl group or a thiopropyl group.

The linker may be (i) HS—(CH₂)₂—CONH-AAY; (ii) HS—(CH₂)₂—CONH-LAAY (SEQ ID NO: 39); (iii) HS—(CH₂)₃—CONH-AAY; (iv) HS—(CH₂)₃—CONH-FLAAY (SEQ ID NO: 38); (v) HS—(CH₂)₁₀—(CH₂OCH₂)₇—CONH-AAY; and (vi) HS—(CH₂)₁₀—(CH₂OCH₂)₇—CONH-FLAAY (SEQ ID NO: 38). In this case, the thiol group of the non-peptide portion of the linker links the linker to the core.

Other suitable linkers for attaching a peptide or polynucleotide to a nanoparticle are known in the art, and may be readily identified and implemented by the skilled person.

As explained above, the pharmaceutical composition may comprise multiple immunogenic peptides or multiple polynucleotides. When the pharmaceutical composition comprises more than one immunogenic peptide or polynucleotide, two or more (such as three or more, four or more, five or more, ten or more, or twenty or more) of the immunogenic peptides or polynucleotides may be attached to the same nanoparticle. Two or more (such as three or more, four or more, five or more, ten or more, or twenty or more) of the immunogenic peptides or polynucleotides may each be attached to different nanoparticle. The nanoparticles to which the immunogenic peptides or polynucleotides are attached may though be the same type of nanoparticle. For instance, each immunogenic peptide or polynucleotide may be attached to a gold nanoparticle or gold glycol-nanoparticle. Each immunogenic peptide or polynucleotide may be attached to a CaP nanoparticle. The nanoparticle to which the immunogenic peptides or polynucleotides are attached may be a different type of nanoparticle. For instance, one immunogenic peptide or polynucleotide may be attached to a gold nanoparticle, and another immunogenic peptide or polynucleotide may be attached to a CaP nanoparticle.

Compositions may be prepared together with a physiologically acceptable carrier or diluent. Typically, such compositions are prepared as liquid suspensions of peptides and/or peptide-linked nanoparticles.

### Medicaments, Methods and Therapeutic Use

The invention provides a method of preventing or treating a common cold coronavirus (CCC) infection in an individual, comprising administering the pharmaceutical composition of the invention to the individual. The invention also provides a pharmaceutical composition of the invention for use in a method of preventing or treating a common cold coronavirus infection in an individual, the method comprising administering the pharmaceutical composition to the individual.

Typically, the individual is human. The individual may, however, be an animal such as a dog, cat, rabbit, guinea pig, horse, bovine, sheep, goat, bird, bat and so on.

The common cold coronavirus infection may be caused by an endemic common cold coronavirus. For example, the common cold coronavirus infection may be a 229E infection. The common cold coronavirus infection may be a NL63 infection. The common cold coronavirus infection may be an OC43 infection. The common cold coronavirus infection may be an HKU1 infection. Preferably, the common cold coronavirus infection is a 229E infection.

The pharmaceutical composition may comprise a pharmaceutically acceptable carrier or diluent. The pharmaceutical composition may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA.

The pharmaceutical composition may be administered by any route. Suitable routes include, but are not limited to, the intravenous, intramuscular, intraperitoneal, subcutaneous, intradermal, transdermal and oral/buccal routes.

The peptides, peptide-linked nanoparticles, polynucleotides, or polynucleotide-linked nanoparticles are administered in a manner compatible with the dosage formulation and in such amount will be therapeutically effective. The quantity to be administered depends on the subject to be treated, the virus to be prevented or treated, the purpose of administration (i.e. prevention or treatment of disease), and the capacity of the subject's immune system. Precise amounts of peptides, polynucleotides or nanoparticles required to be administered may depend on the judgement of the practitioner and may be peculiar to each subject.

Any suitable number of peptides, peptide-linked nanoparticles, polynucleotides, or polynucleotide-linked nanoparticles may be administered to a subject. For example, at least, or about, 0.2×10⁶, 0.25×10⁶, 0.5×10⁶, 1.5×10⁶, 4.0×10⁶ or 5.0×10⁶ peptides, peptide-linked nanoparticles, polynucleotides, or polynucleotide-linked nanoparticles per kg of patient may administered. For example, at least, or about, 10⁵, 10⁶, 10⁷, 10⁸, 10⁹ peptides, peptide-linked nanoparticles, polynucleotides, or polynucleotide-linked nanoparticles may be administered. As a guide, the number of peptides, peptide-linked nanoparticles, polynucleotides, or polynucleotide-linked nanoparticles to be administered may be from 10⁵ to 10⁹, preferably from 10⁶ to 10⁸.

### Complexes

The invention provides a complex comprising a peptide of the invention bound to a MHC molecule. The complex may therefore comprise, for example, a peptide comprising sequence selected from SEQ ID NOs: 1 to 37 or a variant of any thereof bound to a MHC molecule. Such peptides are described in detail above. Any of the aspects described above in connection with the peptide of the invention may apply to the complex of the invention.

Peptide: MHC binding is well-known in the art. Preferably, the binding between the peptide(s) and MHC molecule(s) comprised in the complex is non-covalent. The binding may be mediated by, for example, electrostatic interaction, hydrogen bonds, van der Waals forces and/or hydrophobic interactions.

The MHC molecule may be a MHC class 1 molecule or a MHC class II molecule. Preferably, the MHC molecule is a MHC class I molecule. The MHC molecule may be of any HLA supertype. For example, the MHC class I molecule may be of supertype A1, A10, A11, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, or C9.

The complex may comprise two or more peptides of the invention, and two or more MHC molecules. For example, the complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention. The complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules. The complex may, for example, comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules respectively. The complex may comprise the same number of peptides of the invention as MHC molecules. The complex may comprise a different number of peptides of the invention from the number MHC molecules. The complex may, for example, comprise four MHC molecules. The complex may comprise or consist of a MHC tetramer. The complex may, for example, comprise twelve MHC molecules. The complex may comprise or consist of a MHC dodecamer.

When the complex comprises two or more peptides of the invention, each of the two or more peptides may be the same. Alternatively, each of the two or more peptides may be different. When the complex comprises three or more peptides of the invention, each of the three or more peptides may be the same. When the complex comprises three or more peptides of the invention, each of the three or more peptides may be different. When the complex comprises three or more peptides of the invention, some of the three or more peptides may be the same and some of the three of more peptides may be different.

When the complex comprises two or more MHC molecules, each of the two or more MHC molecules may be the same. Alternatively, each of the two or more MHC molecules may be different. When the complex comprises three or more MHC molecules, each of the three or more MHC molecules may be the same. When the complex comprises three or more MHC molecules, each of the three or more MHC molecules may be different. When the complex comprises three or more MHC molecules, some of the three or more MHC molecules may be the same and some of the three of more MHC molecules may be different.

When the complex comprises two or more peptides of the invention and two or more MHC molecules, each peptide may be bound to one of the two or more MHC molecules. That is, each peptide comprised in the complex may be bound to an MHC molecule comprised in the complex. Preferably, each peptide comprised in the complex is bound to a different MHC molecule comprised in the complex. That is, each MHC molecule comprised in the complex is preferably bound to no more than one peptide comprised in the complex. The complex may, however, comprise one or more peptides of the invention that are not bound to an MHC molecule. The complex may comprise one or more MHC molecules that are not bound to a peptide of the invention.

The MHC molecule or molecules comprised in the complex may be linked to one another. For example each of the one or more MHC molecules in the complex may be attached to a backbone molecule or a nanoparticle. The MHC molecule or molecules comprised in the complex may be attached to a dextran backbone. That is, the complex may comprise or consist of an MHC dextramer. Mechanisms for attaching a MHC molecule or molecules to a dextran backbone are known in the art. Any number of MHC molecules may be attached to the dextran backbone. For example, one or more, two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules may be attached to the dextran backbone.

The complex may comprise a fluorophore. Fluorophores are well-known in the art and include FITC (fluorescein isothiocyanate), PE (phycoerythrin) and APC (allophycocyanin). The complex may comprise any number of fluorophores. For example, the complex may comprise two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more fluorophores. When the complex comprise multiple fluorophores, the fluorophores comprised in the complex may be the same or different. When the complex comprises a backbone, such as a dextran backbone, the fluorophore is preferably attached to the dextran backbone. Mechanisms for attaching a fluorophore to a dextran backbone are known in the art.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

The following examples illustrate the invention.

### Example 1

### Introduction

Human coronavirus 229E (HcoV-229E) belongs to one of the seven human coronaviruses which include MERS-CoV, SARS-CoV-1 and SARS-CoV-2. It can infect humans and bats. The current project was designed to identify MHC class I peptides in post-HcoV-229E infection cell pellet and whole cell lysate post infection. HepG2 cells were infected, harvested, lysed and MHC class I peptides were extracted to generate peptide sample mixture for mass spectrometry analysis. Additionally, a control sample (mock infection) was collected and processed following the same procedure or infection sample. An ultra high pressure liquid chromatography (UPLC) system coupled with an Eclipse triploid mass spectrometer was used for peptide separation and identification. One collision mode (CID) were applied in mass spec analysis of infection samples. A data dependent acquisition (DDA) method was implemented for real time sample acquisition. The generated datasets were processed using SEQUEST HT software for the database searches and comparison.

### Methods

### 1. HcoV-229E Infected HepG2 Cell Preparation (Performed by GMU)

HepG2 cells (2×10⁷ total cells) were grown and infected with HcoV-229E (MOI=0.1) for 24 h.

### 2. HcoV-229E MHC Class I Peptide Discovery Using Mass Spectrometry Sample Analysis

a) Sample preparation: HcoV-229E strain infected HepG2 cells were harvested and washed by PBS once. The cell pellet was treated with 0.1% TFA and the supernatant was collected (provided by GMU). The cell pellet was treated with lysis buffer and processed (provided by GMU) using the immunoproteomic protocol to isolate and purify MHC class I peptides. The two samples (samples from steps 1&2) were pooled, cleaned up and fractionated on an offline HPLC system. Each fraction was analyzed on a UPLC-Nano-MS/MS system.
b) DDA mode: Samples were analyzed using an Eclipse triploid mass spectrometer coupled to a Dionex Ultimate 3000 RLSCnano System (Thermo Scientific). Samples were loaded on to a fused silica trap column Acclaim PepMap 100, 75 um×2 cm (ThermoFisher). After washing with 0.1% TFA, the trap column was brought in-line with an analytical column (Nanoease MZ peptide BEH C18, 130A, 1.7 um, 75 um×250 mm, Waters) for LC-MS/MS. Peptides were eluted using a segmented linear gradient from gradient 4-15% B in 30 min (where A: 0.2% formic acid, and B: 0.16% formic acid, 80% acetonitrile), 15-25% B in 40 min, 25-50% B in 44 min, and 50-90% B in 11 min. Solution B then returns at 4% for 5 minutes for the next run. Mass spectrometry data (CID mode) was acquired using a data-dependent acquisition procedure with a cyclic series of a full scan from 375-1500 with resolution of 240,000 normalized (AGC) target 250% of normal (1E6), maximum injection time 50 ms. The top S (1 sec) and dynamic exclusion of 30 sec were used for selection of parent ions of charge stage 2-7 for MSMS with intensity threshold at 5E3. The selected ions were transmitted to ion trap with isolation window 1.2 m/z normalized AGC target 200% and fragmented with relative collision energy 35% and scanned in the ion trap with rapid scan rate. Mass spectrometry data was acquired using a data-dependent acquisition procedure with a cyclic series of a full scan from 350-1500 with resolution of 120,000 control (AGC) target 1E6, maximum injection time 100 ms.

### 3. Database Search

Database searching of all raw spectra files was performed using Proteome Discoverer 1.4 (Thermo Fisher Scientific). SEQUEST was used for database searching against HcoV 229E protein sequence databases 1) positive sense translations 2) negative sense translations and 3) IPI human database. Database searching against the corresponding reverse database was also performed to evaluate the false discovery rate (FDR) of peptide/protein identification. The database searching parameters included up to two missed cleavages for no enzyme digestion, precursor mass tolerance 10 ppm, product ion mass tolerance 0.4 Da, and methionine oxidation as variable modifications. The result from each run was filtered with peptide confidence value as high to obtain FDR less than 1% on peptide level. On the protein level the search parameters included minimum number of peptide 1 for each protein, count only rank 1 peptides, and count peptide only in top scored proteins were applied for all data filtration. In addition, protein grouping was enabled, and strict maximum parsimony principle was applied. Afterwards, manual evaluation was applied into each mass spectra.

### Results

TABLE 4
HcoV-229E ligandome summary

| Sample | Control | HcoV-229E(CID) |
|---|---|---|
| Self-derive peptide (full | 31719 | 22795 |
| list)^{a} | | |
| Self-derive protein (full | 11638 | 10097 |
| list) | | |
| 229E viral peptide | 23 | 37 |
| (full list) | | |

Notes:
^{a}full list generated by pre-screening criteria (confidence-high, amino acid length-8 to 15 aa, rank: 1, XCorr >1.5 for charge 2, XCorr >2 for charge 3)

TABLE 5
List of MHC class I peptides from HcoV-229E viral proteins

| SEQ | | Protein source | Viral |
|---|---|---|---|
| ID NO: | Sequence | NCBI ref. seq. | origin |
| 1 | AMLKCVAFCDE | NCBI ref. seq. NP_073549.1 | 229E |
| 2 | ANGCSTIAQAV | NCBI ref. seq. NP_073549.1 | 229E |
| 3 | AQGVFGVNM | NCBI ref. seq. NP_073549.1 | 229E |
| 4 | ARLEPCNGTDID | NCBI ref. seq. NP_073549.1 | 229E |
| 5 | AVTTGDVKIM | NCBI ref. seq. NP_073549.1 | 229E |
| 6 | DIVVVDEVSMCTNYD | NCBI ref. seq. NP_073549.1 | 229E |
| 7 | DSLCAKAVTAY | NCBI ref. seq. NP_073549.1 | 229E |
| 8 | EDFLNMDIGVFIQ | NCBI ref. seq. NP_073549.1 | 229E |
| 9 | EVNADIVVVDEVSMC | NCBI ref. seq. NP_073549.1 | 229E |
| 10 | FVGADGELPV | NCBI ref. seq. NP_073549.1 | 229E |
| 11 | FVKSICNSAVAV | NCBI ref. seq. NP_073549.1 | 229E |
| 12 | FYCTNNTLVSGDAHI | NCBI ref. seq. NP_073549.1 | 229E |
| 13 | GAKVVNANVLTK | NCBI ref. seq. NP_073549.1 | 229E |
| 14 | GYIADISAF | NCBI ref. seq. NP_073549.1 | 229E |
| 15 | IACSKSARLKRFPVN | NCBI ref. seq. NP_073549.1 | 229E |
| 16 | IADFLAGSSDV | NCBI ref. seq. NP_073549.1 | 229E |
| 17 | IFAQTSDTA | NCBI ref. seq. NP_073549.1 | 229E |
| 18 | IVQMIADFLA | NCBI ref. seq. NP_073549.1 | 229E |
| 19 | KFLNAFDVFVTAIQ | NCBI ref. seq. NP_073549.1 | 229E |
| 20 | LIAGKLLPPV | NC_002645_Human_coronavirus | 229E |
| | | _229E_Translation_RF-1_24 | |
| 21 | LNCALGAFAIFCC | NCBI ref. seq. NP_073549.1 | 229E |
| 22 | MHGVTLKI | NCBI ref. seq. NP_073549.1 | 229E |
| 23 | MKVKATKGEGDGGI | NCBI ref. seq. NP_073549.1 | 229E |
| 24 | NAMLKCVAF | NCBI ref. seq. NP_073549.1 | 229E |
| 25 | NEADYRCACYA | NCBI ref. seq. NP_073549.1 | 229E |
| 26 | PNLNLGILQVT | NC_002645_Human_coronavirus | 229E |
| | | _229E_Translation_RF+1_20 | |
| 27 | PSLVMPPSPSPLV | NC_002645_Human_coronavirus | 229E |
| | | _229E_Translation_RF-3_42 | |
| 28 | QAAAAMYKEARAVN | NCBI ref. seq. NP_073549.1 | 229E |
| 29 | QTSQALQTVATALNK | NCBI ref. seq. NP_073549.1 | 229E |
| 30 | SEISANGCSTIAQA | NCBI ref. seq. NP_073549.1 | 229E |
| 31 | SNFNTLFATTIPN | NCBI ref. seq. NP_073549.1 | 229E |
| 32 | TIQGPPGSGKS | NCBI ref. seq. NP_073549.1 | 229E |
| 33 | TNVPLQVGFSNG | NCBI ref. seq. NP_073549.1 | 229E |
| 34 | VGGTIQIL | NCBI ref. seq. NP_073549.1 | 229E |
| 35 | VLFSATAVKTGGK | NCBI ref. seq. NP_ 073549.1 | 229E |
| 36 | VLNNGFGGKQI | NCBI ref. seq. NP_073549.1 | 229E |
| 37 | VTSGLGTVDADY | NCBI ref. seq. NP_073549.1 | 229E |

SEQ ID NOs: 1 to 19, 21 to 26, and 28 to 37 are MHC class I presented epitopes from 229E common cold coronavirus. SEQ ID NOs: 20 (LIAGKLLPPV) and 27 (PSLVMIPPSPSPLV) are reverse peptides from the common cold coronavirus 229E. That is, SEQ ID NOs: 46 (LIAGKLLPPV) and 53 (PSLVMIPPSPSPLV) are epitopes from a polypeptide encoded by an open reading frame (ORF) encoded by at least part of the genome of a 229E common cold coronavirus in the opposite sense to positive sense RNA capable of translation.

By running the SYFPEITHI epitope binding prediction program, SEQ ID NO: 20 (LIAGKLLPPV); was predicted to have the following HILA binding: A2:29.

1. A peptide comprising a sequence selected from SEQ ID NOs: 1 to 37 or a variant of any thereof.

2. The peptide of claim 1, wherein the variant differs from the corresponding sequence selected from SEQ ID NOs: 1 to 37 by a single amino acid.

3. The peptide of claim 1 or 2, wherein the variant differs from the corresponding sequence selected from SEQ ID NOs: 1 to 37 by a single amino acid substitution, insertion or deletion.

4. The peptide of claim 4, wherein the substitution is a conservative substitution.

5. The peptide of any one of the previous claims, which is 8 to 30 amino acids in length.

6. A polynucleotide encoding the peptide of any one of the preceding claims.

7. The polynucleotide of claim 6, wherein the polynucleotide comprises or consists of RNA, optionally mRNA.

8. A pharmaceutical composition comprising a peptide according to any one of claims 1 to 5.

9. The pharmaceutical composition of claim 8, wherein the peptide interacts with at least two different HLA supertypes.

10. The pharmaceutical composition of claim 8 or 9, which comprises two or more peptides according to any one of claims 1 to 5, each comprising a different sequence selected from SEQ ID NOs: 1 to 37 or a variant thereof.

11. The pharmaceutical composition of claim 10, wherein each of the two or more peptides interacts with a different HLA supertype.

12. A pharmaceutical composition comprising a polynucleotide according to claim 6 or 7.

13. The pharmaceutical composition of claim 12, wherein the peptide encoded by the polynucleotide interacts with at least two different HLA supertypes.

14. The pharmaceutical composition of claim 13, which comprises two or more polynucleotides according to claim 6 or 7, each encoding a peptide that comprises a different sequence selected from SEQ ID NOs: 1 to 37 or a variant thereof.

15. The pharmaceutical composition of claim 14, wherein each of the peptides encoded by the two or more polynucleotides interacts with a different HLA supertype.

16. The pharmaceutical composition of any one of claims 8 to 15, wherein the peptide or polynucleotide comprised in the composition is attached to a nanoparticle, optionally via a linker.

17. The pharmaceutical composition of claim 16, wherein the nanoparticle is a gold nanoparticle, a calcium phosphate nanoparticle, or a silicon nanoparticle, optionally wherein the gold nanoparticle is coated with alpha-galactose and/or beta-GlcNAc.

18. A method of preventing or treating a common cold coronavirus (CCC) infection in an individual, comprising administering the pharmaceutical composition of any one of claims 8 to 17 to the individual.

19. The pharmaceutical composition of any one of claims 8 to 17 for use in a method of preventing or treating a common cold coronavirus (CCC) infection in an individual, the method comprising administering the pharmaceutical composition to the individual.

20. The use of the pharmaceutical composition of any one of claims 8 to 17 in the preparation of a medicament for use in a method of preventing or treating a common cold coronavirus (CCC) infection in an individual.

21. The method of claim 18, or the composition for use of claim 19, or the use of claim 20, wherein the common cold coronavirus is human coronavirus 229E.

22. A complex comprising a peptide according to any one of claims 1 to 5 bound to a MHC molecule.

23. A complex according to claim 22, wherein the complex comprises two or more peptides according to any one of claims 1 to 5 and two or more MHC molecules, optionally wherein each peptide is bound to one of the two or more MHC molecules.

24. A complex according to claim 23, wherein each of the two or more MHC molecules is attached to a dextran backbone.

25. A complex according to claim 24, wherein the complex further comprise a fluorophore, optionally wherein the fluorophore is attached to the dextran backbone.
